(19) United States
(12) Patent Application Publication
(10) Pub. No.: US 2026/0286269 A1
(43) Pub. Date: Sep. 24, 2026

(54) MANUFACTURING METHOD WITH FEEDBACK CONTROL

(71) Applicant: Chr. Hansen A/S, Hoersholm (DK)

(72) Inventors: Karsten HELLMUTH, Nienburg/Weser (DE); Bastian BARTLING, Nienburg/Weser (DE); Philipp Paul GRUENERT, Nienburg/Weser (DE)

(73) Assignee: Chr. Hansen A/S, Hoersholm (DK)

(21) Appl. No.: 19/470,028

(22) PCT Filed: Mar. 27, 2024
(86) PCT No.: PCT/EP2024/058325

§ 371 Date: Sep. 27, 2025

(30) Foreign Application Priority Data
Mar. 31, 2023 (EP) 23165791.7

(51) Int. Cl. C12M 1/00; B01D 61/14; B01D 61/22; C12M 1/34

(52) U.S. Cl. CPC C12M 29/04; B01D 61/147; B01D 61/22; C12M 41/46; B01D 2311/246

(57) ABSTRACT

The present disclosure generally relates to a method of manufacturing biological product using fermentation and subsequent purification of the biological product, such as an enzyme. By utilization of online biomass data for a feedback control in the fermenter, an efficient and stable cross-flow microfiltration operation can be performed with active cells.

### FIELD

The present disclosure generally relates to a method of manufacturing biological product using fermentation and subsequent purification of the biological product.

### BACKGROUND

Manufacturing products by microbial expression through fermentation is a common way of producing various biological substances, such as enzymes. This is for example described in patent publication WO2021170799A1.

After a fermentation is completed, the desired product needs to be isolated from the fermentation broth. This is typically done by first separating the aqueous phase from the cell material in a filtration step, followed by an extraction or adsorption of the product from the filtrate. Often, however, such a filtration step is accompanied by a significant loss of desired product. This is mainly because the filter cake cannot be washed out sufficiently and consequently a rather large amount of the product remains in the filter cloth. In practice, it is observed that the efficiency and the capacity of the filtration process strongly depend on the quality of the fermentation broth.

In order to overcome the above problems in the handling of fermentation broths, patent publication U.S. Pat. No. 651,511B1 describes other filtration methods, such as membrane filtration.

However, such cross-flow filtration methods have disadvantages, primarily from biomass concentration increase rapidly in a non-controlled manner. At a critical cell concentration, the microfiltration membrane would block and disturb the recovery process.

Thus, there is a need for improved manufacturing processes.

### SUMMARY

The present disclosure provides an improved manufacturing process for extracting product from a fermentation broth using a cross-filtration unit, combined with an online biomass probe and a feedback loop for continuous dilution of the fermentation broth.

This is advantageous in several ways, for example by enabling operation at the highest possible biomass concentration, giving a high product concentration in the fermentate/retentate. A high product concentration in the retentate is also enabled, providing a high product titer in the permeate. This leads to faster and more efficient cross flow operation with a good recovery yield. Also, the product, such as an enzyme, is secreted into the fermentation broth in a fed-batch fermentation setup. The product, such as an enzyme, is realized by crossflow microfiltration whilst fed-batch fermentation process is kept running to avoid lysis and contamination with unwanted enzymes, such as proteases.

According to a first aspect of the invention, a process for producing a product by fermentation of a host cell is disclosed, comprising the steps of
a) adding host cell culture to a fermenter;
b) feeding water of buffer to the fermenter from a buffer tank, operably connected to the fermenter through a pump or valve;
c) feeding fermentate from the fermenter to a cross-flow microfiltration unit;
d) feeding biomass back to the fermenter from the cross-flow microfiltration unit; and
e) feeding permeate from the cross-flow microfiltration unit to a product vessel; wherein a biomass probe is in the fermenter, said biomass probe measuring cell concentration a process control system receives data about cell concentration and controls the pump or valve feeding water or buffer into the fermenter so that a certain predetermined cell concentration is maintained.

In an embodiment, the process further comprising a feed medium tank, operably connected to the fermenter.

According to a second aspect of the invention, a product obtainable by the process according to the first aspect is disclosed.

In an embodiment, the product obtainable by the process according to the first aspect is an enzyme.

In yet another embodiment, the product obtainable by the process according to the first aspect is a naturally occurring enzyme or enzymes produced by native microorganisms or expression hosts.

### BRIEF DESCRIPTION OF THE FIGURES

FIG. 1 is a schematic representation of an embodiment.

FIG. 2 is a graph showing online and offline biomass according to an embodiment.

FIG. 3 is a graph showing online and offline biomass according to an embodiment.

FIG. 4 is a graph showing that the dilution rate according to an embodiment.

FIG. 5 is a graph showing product concentration according to an embodiment.

FIG. 6 is a graph showing online and offline biomass according to an embodiment.

FIG. 7 is a graph showing fermenter volume and water addition according to an embodiment

FIG. 8 is a graph showing product concentration according to an embodiment.

FIG. 9 is a schematic representation of an embodiment.

### DETAILED DESCRIPTION OF THE INVENTION

This disclosure relates to an efficient product recovery from a high cell density fermentation using cross flow microfiltration. In one embodiment, the product is an enzyme.

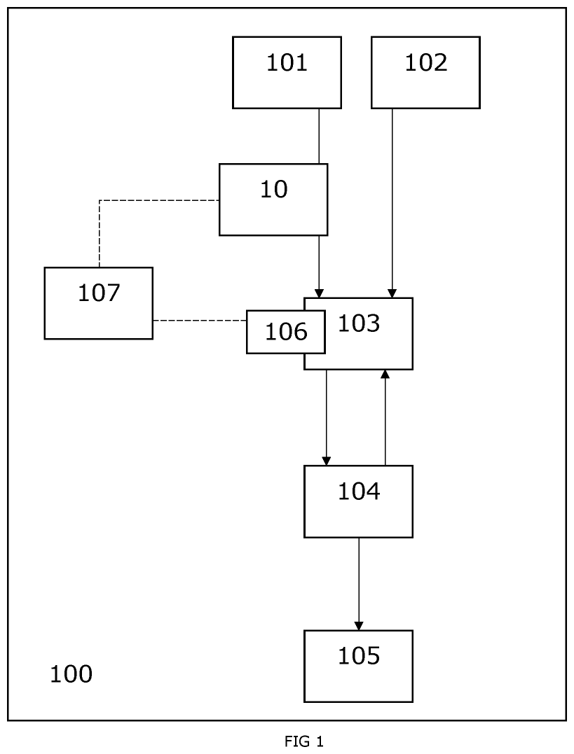

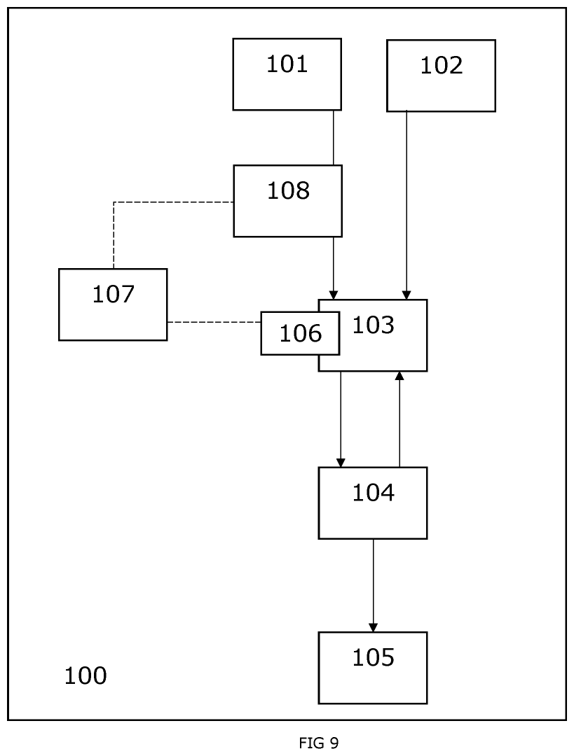

An integrated up-and downstream process was developed. FIGS. 1 and 9 shows a schematic overview of the process setup (100). Water or buffer is kept in a buffer tank (101) and feed medium solution is kept in a feed medium tank (102). Both buffer/water and feed medium solution, such as sugar solution, are fed into the fermenter (103), where the cells are proliferating and product is expressed. From the fermenter, a feed is directed to a cross-flow microfiltration unit (104), where permeate is separated from biomass. The biomass is fed back to the fermenter (103), and the permeate is fed into a product vessel (105). Thus, the crossflow microfiltration unit is used to separate the enzyme from the cell containing fermentation broth.

In order to avoid cell lysis in the fermenter (103) and thereby the release of proteases and other by-products, cultivation is continued while the harvesting of product from the fermentation broth has started. Product stability in the fermenter (103) is obtained by controlling pH, temperature, and oxygen supply in the desired range by connecting the fermenter and cross flow filtration unit directly.

Furthermore, to enable control of the biomass in the highest possible concentration, an online biomass probe (106) is used in the fermenter (103), to measure biomass concentration. The biomass probe (106) sends data to a process control system (107), which interprets the data. A feedback loop is created when the process control system (107) regulates a pump or valve (108), which supplies water or buffer from the buffer tank (101) into the fermenter (103). This enables continuous dilution of the cell-containing broth, based on the data from the biomass probe (106).

Because of this feedback control, the biomass concentration can be kept at an optimal level. Otherwise, the biomass concentration would increase rapidly in a non-controlled manner. At a critical cell concentration, the microfiltration membrane would block and disturb the recovery process.

Further advantages of the online biomass feedback control are many. For example, this solution enables operation at the highest possible biomass concentration, giving a high product concentration in the fermentate/retentate. It also enables a high product concentration in the retentate, providing a high product titer in the permeate. This leads to faster and more efficient cross flow operation with a good recovery yield. Also, the product, such as an enzyme, is secreted into the fermentation broth in a fed-batch fermentation setup. The product, such as an enzyme, is realized by crossflow microfiltration whilst fed-batch fermentation process is kept running to avoid lysis and contamination with unwanted enzymes, such as proteases.

In an embodiment, cell concentration measured by the biomass probe (106) can also be compared to the biomass content (packed cell volume (PCV), vol %) estimated in an offline sample.

In one embodiment, the product is an enzyme. The application of continuous harvesting of enzyme product using the process setup (100) according to an embodiment, is particularly advantageous since enzyme products typically cannot be present during deactivation of the GMO production host strain, since they either are unstable at the necessary pH and/or temperature. Further advantages of the process setup (100) according to an embodiment, are minimizing release of proteases by cellular lysis and minimizing release of GMO DNA by cellular lysis thus mitigating the need for a separate, elaborate DNA capture process. A further advantage with the process setup (100) according to an embodiment is that efficiency increases by continuous cross-flow-filter operation at optimal process parameters, since membrane fouling by biomass getting stuck and blocking the cross-flow filter is minimized and controlled. Resulting higher product titers at reduced permeate volumes may avoid the need to concentrate by ultrafiltration and enzyme capturing chromatography.

### Cloning and Expression

Any product suitable for microbial expression might be produced using the process setup (100) according to an embodiment. In patent publication WO2021170799A1, a specific system for expressing enzyme is disclosed.

Suitable expression hosts are readily known to a person skilled in the art. Examples of expression hosts are Pichia pastoris, or Saccharomyces spp., Saccharomyces cerevisiae, Schizosaccharomyces spp., Candida spp., Candida cylindracea, Kluyveromyces spp., Hansenula polymorpha, Fusarium spp., Fusarium oxysporium, Aspergillus spp., Aspergillus oryzae or Aspergillus niger, Trichoderma spp., Escherichia coli or Bacillus spp., Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces spp., Streptomyces lividans or Streptomyces murinus, Corynebacterium spp., Yarrowia, preferably Aspergillus oryzae, Aspergillus niger, or Bacillus subtilis.

Recombinant cloning and expression system, suitable for expression of the desired product, may be further introduced in a host cell, such as a recombinant host cell, using standard transformation or transfection protocols known to the person skilled in the art and explained in literature (for example, in J. Sambrook, E. F. Fritsch, and T. Maniatis, 1989, Molecular Cloning: A Laboratory manual, 2nd edition, books 1-3, Cold Spring Harbor Laboratory Press).

### EXAMPLES

### Example 1

In a first experiment biomass concentration was measured online (in pF/cm) and offline (PCV in vol %). The loss of fermentation volume by permeate flow was adapted manually with the addition of water trying to keep the fermenter volume constant. Without automatic control a blockage of the microfiltration membrane could be prevented, but no stable biomass concentration was obtained as crossflow parameters (trans-membrane pressure, CF overflow, permeate flow) oscillate and biomass concentration increased.

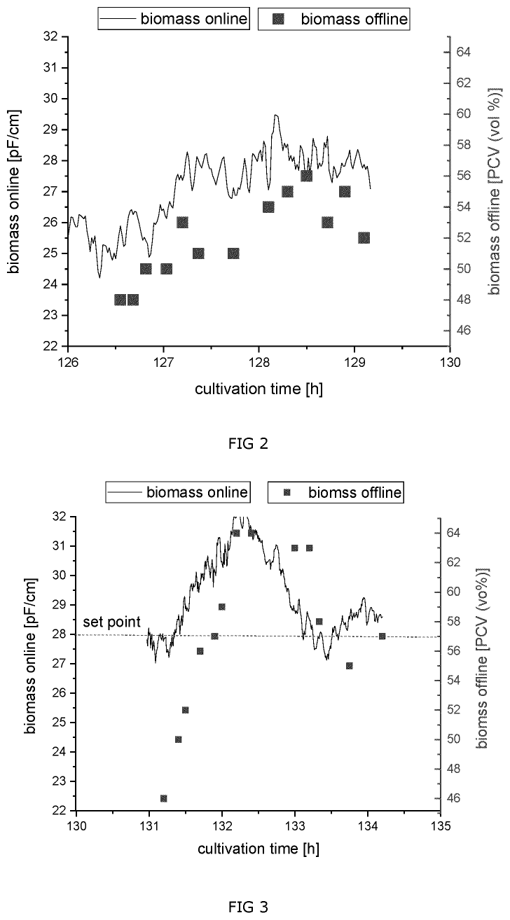

FIG. 2 is a graph showing online and offline biomass (as PCV, packed cell volume) measurement in fermenter during a manually controlled cross flow microfiltration process. Efficient penetration of Enzyme through membrane is dependent on biomass concentration and biofouling of membranes.

### Example 2

A second experiment shows that too high biomass concentration inhibits penetration of product into the permeate.

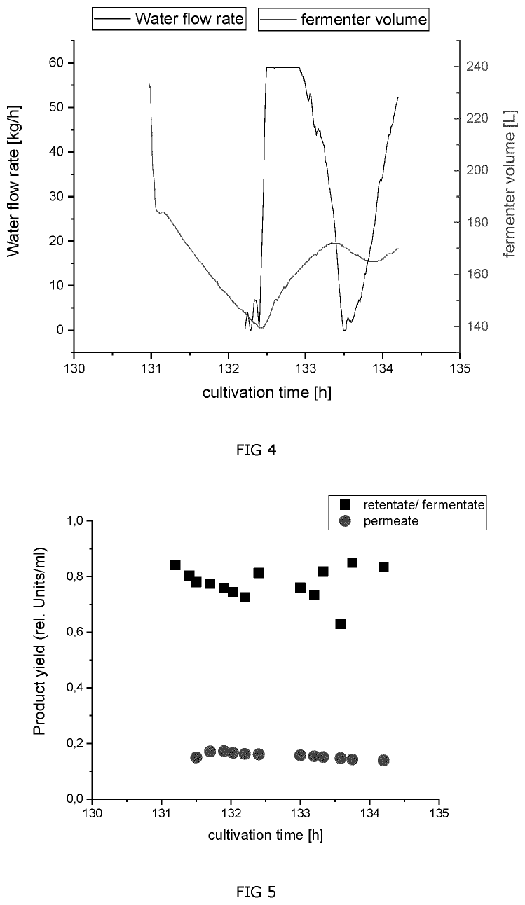

FIG. 3 is a graph showing online and offline biomass measurement in fermenter during a PID controlled cross flow microfiltration process; set-point for controller was 28 pF/cm. FIG. 4 is a graph showing fermenter volume and water addition over cultivation time.

The feedback loop allows a flexible adaption to the biomass concentration effected by the extraction of permeate and continued growth. The increase in fermenter. Is due to more volume being added via feed and water dilution than removed via permeate.

FIG. 4 is a graph showing that the dilution rate altered dynamically over the course of the enzyme DSP. The controller was started too late thus the dilution was too slow and the setpoint of 28 pF/cm was achieved late.

As consequence, extremely high biomass overshoot (>30 pF/cm, PCV of 60 vol %) caused a biofouling of the membrane and reduced the enzyme penetration. This can be seen in FIG. 5.

FIG. 5 is a graph showing product concentration in retentate (fermentate) and in the permeate. It can be seen that there is low penetration through the cross-flow membrane.

### Example 3

A third experiment describes an ideal DSP. As setpoint, a biomass concentration of 26 pF/cm was chosen. PID controller parameters were optimized to allow for this setpoint to be kept by ±0.37 pF/cm. The corresponding PCV value was in a range of 51.7±1.0 vol %.

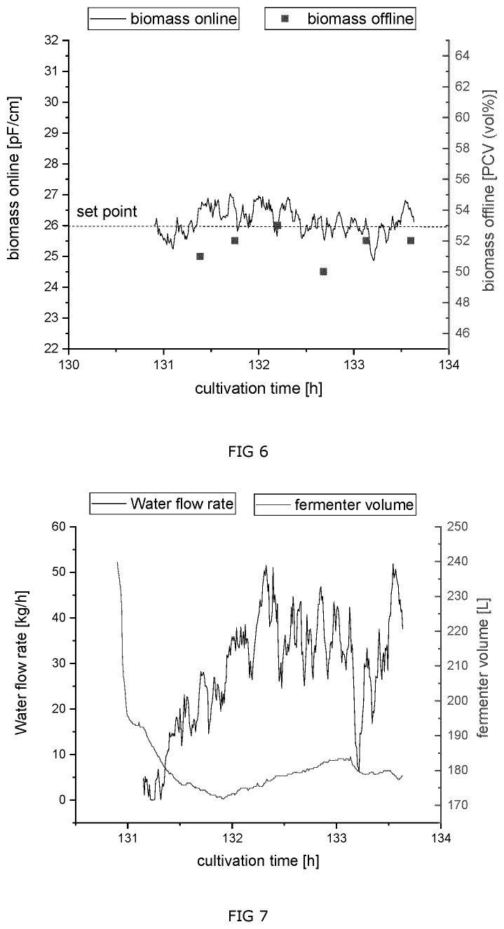

FIG. 6 is a graph showing online and offline biomass measurements in fermenter during a PDI controlled cross flow microfiltration process; setpoint for controller was 26 pF/cm.

FIG. 7 is a graph showing fermenter volume and water addition as over cultivation time.

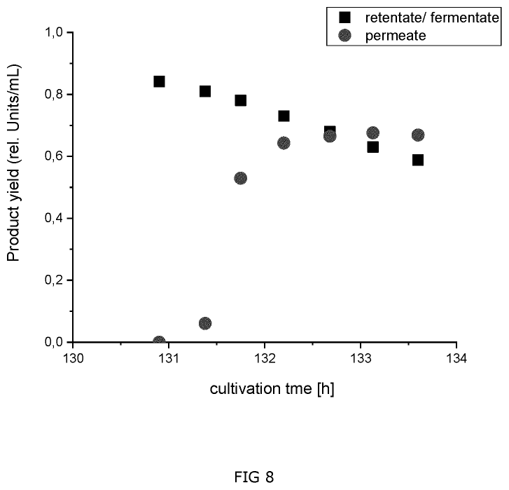

FIG. 8 is a graph showing product concentration in retentate (fermentate) and in the permeate. Optimal penetration through the crossflow membrane is apparent.

The dynamic dilution rate secured a stable CFF operation with high enzyme penetration (and in a high product concentration) over several hours.

Biomass viability is ensured by continued fed-batch fermentation during enzyme harvest in-turn minimizing cell lysis, protease release, and cross contamination of the product. A dilution of biomass to an optimal value by water addition is controlled on-line in reference to a Hamilton InCyte signal in the fermenter securing a stable in-situ downstream processing. Thereby membrane fouling is controlled by building up a defined filter cake of cells resulting in a low transmembrane retention of the target enzyme—too high cell densities block the filtration membrane.

By utilization of online biomass data for a feedback control in the fermenter an efficient and stable cross-flow microfiltration operation can be performed with active cells. This is advantageous, because product is obtained in a high concentration and without significant by-products.

The process set-up according to an embodiment, can be used for several enzymes without additional purification and concentration steps.

The present invention has been described with reference to various embodiments, aspects, examples, or the like. It is not intended that these elements be read in isolation from one another. Thus, the present disclosure provides for the combination of two or more of the embodiments, aspects, examples, or the like.

All embodiments described herein are intended to be within the scope of the invention disclosed. These and other embodiments of the present invention will become readily apparent to those skilled in the art from the following detailed description of the preferred embodiments having reference to the whole description, the invention not being limited to any preferred embodiment(s) disclosed.

1. A process for producing a product by fermentation of a host cell, said process comprising the steps of:
a) adding host cell culture to a fermenter;
b) feeding water or buffer to the fermenter from a buffer tank, operably connected to the fermenter through a pump or valve;
c) feeding fermentate from the fermenter to a cross-flow microfiltration unit;
d) feeding biomass back to the fermenter from the cross-flow microfiltration unit; and
e) feeding permeate from the cross-flow microfiltration unit to a product vessel;
wherein a biomass probe is in the fermenter, said biomass probe measuring cell concentration a process control system receives data about cell concentration and controls the pump or valve feeding water or buffer into the fermenter so that a certain predetermined cell concentration is maintained.

2. The process according to claim 1, further comprising a feed medium tank, operably connected to the fermenter.

3. A product obtained by the process according to claim 1.

4. The process according to claim 1, wherein the product is an enzyme.

5. The process according to claim 4, wherein the product is a naturally occurring enzyme and/or an enzyme produced by native microorganisms.
